(19) United States
(12) Patent Application Publication
(10) Pub. No.: US 2020/0138727 A1
(43) Pub. Date: May 7, 2020

(54) ENHANCED THERAPEUTIC AGENT AND NUCLEIC ACID DELIVERY VIA HDL OR RECONSTITUTED HDL NANOPARTICLES

(71) Applicant: UNIVERSITY OF NORTH TEXAS HEALTH SCIENCE CENTER, FORT WORTH, TX (US)

(72) Inventors: ANDRAS G. LACKO, FORT WORTH, TX (US); NIRUPAMA A. SABNIS, FORT WORTH, TX (US); SUNIL SHAH, FORT WORTH, TX (US); LINDA K. MOOBERRY, ARLINGTON, TX (US)

(21) Appl. No.: 16/606,232

(22) PCT Filed: Apr. 20, 2018
(86) PCT No.: PCT/US18/28498

§ 371 Date: Oct. 18, 2019

Related U.S. Application Data
(60) Provisional application No. 62/488,223, filed on Apr. 21, 2017.
(60) Provisional application No. 62/551,944, filed on Aug. 30, 2017.

(51) Int. Cl. A61K 9/51; A61K 31/704; A61K 31/713

(52) U.S. Cl. CPC A61K 9/5123; A61K 31/713; A61K 31/704

(57) ABSTRACT

The present invention provides high density lipoprotein (HDL) or reconstituted HDL (rHDL) particles for the delivery of non-nucleic acid therapeutic agents, therapeutic agents or nucleic acids to cells and tissues. The use of HDL or rHDL particles has advantages over other delivery systems because they are smaller in size and their contents are rapidly internalized by receptors of specific cells, including receptors on the surface of tumor tissue. The HDL or rHDL nanoparticles of the present invention may include a positively charged polyamino acid, which neutralizes the negatively charged nucleic acid, thus allowing for successful incorporation of the nucleic acid into an HDL or rHDL particle. Methods of delivering therapeutic agents to cells and target tissues using the disclosed HDL or rHDL particles are provided as are methods of treating various diseases and disorders.

### CROSS-REFERENCE TO RELATED APPLICATIONS

This application claims the benefit of U.S. Provisional Application Ser. No. 62/488,223, filed Apr. 21, 2017 and Ser. No. 62/551,944, filed Aug. 30, 2017, the disclosures of which are hereby incorporated by reference in their entirety, including all figures, tables and amino acid or nucleic acid sequences.

### FIELD OF THE INVENTION

The present invention relates generally to the fields of drug delivery, molecular biology and therapeutics. More particularly, it concerns high density lipoprotein (HDL) particles or reconstituted HDL (rHDL) particles for the delivery of nucleic acids or other therapeutic agents into cells and tissues, and compositions, methods, and kits that involve the HDL or rHDL particles.

The present invention also relates generally to the fields of drug delivery, molecular biology and therapeutics and concerns high density lipoprotein (HDL) particles or reconstituted HDL (rHDL) particles for the delivery of non-nucleic acid therapeutic agents into cells and tissues, and compositions, methods, and kits that involve the HDL or rHDL particles.

### BACKGROUND OF THE INVENTION

The therapeutic effectiveness of therapeutic agents and nucleic acids, such as genes, gene fragments, antisense nucleic acids, and interference RNA molecules has long been hampered by relatively inefficient delivery systems for these molecules into specific cells and tissues. Macromolecular complexes containing lipids (liposomes) have been utilized as delivery vehicles for therapeutic agents, such as proteins, interleukins, cancer chemotherapeutic agents and antisense oligonucleotides (see, e.g., Chonn and Cullis, 1995; Wang et al., 1996; Lundberg, 1997; Weiner, 1994; Bergers et al., 1993; Tani et al., 1994). Other approaches to improve delivery of therapeutic agents include incorporation of specialized lipids or polyethylene glycol into liposomes for extending the residence time of the particles in the circulation (Wang et al., 1996; Allen, 1994) and the attachment of targeting agents, such as glycolipids, proteins, antigens or antibodies to the liposome complex (Vingerhoeds et al., 1996). Despite these improvements and advances, toxic side effects remain a serious concern (McGuire and Ozols, 1998; Fanning et al., 1993; McGuire et al., 1996; Feenstra et al., 1997). HDL particles for the delivery of nucleic acids into target cells and tissues are disclosed in U.S. Pat. No. 8,734,853, which is hereby incorporated by reference in its entirety. However, these particles exhibit heterogeneity in size and issues with respect to long-term shelf life. Thus, there is a continued need for improvements in techniques and vehicles for the delivery of therapeutic agents, particularly nucleic acids, to target cells or tissues.

### BRIEF SUMMARY OF THE INVENTION

The present invention provides high density lipoprotein (HDL) or reconstituted HDL (rHDL) particles for the delivery of therapeutic agents or nucleic acids to cells and tissues. The use of HDL or rHDL particles has advantages over other delivery systems because they are smaller in size and their contents are rapidly internalized by receptors of specific cells, including receptors on the surface of tumor tissue. The HDL or rHDL nanoparticles of the present invention may include a positively charged polyamino acid, which neutralizes the negatively charged nucleic acid, thus allowing for successful incorporation of the nucleic acid into an HDL or rHDL particle. Methods of delivering therapeutic agents to cells and target tissues using the disclosed HDL or rHDL particles are provided as are methods of treating various diseases and disorders.

Another embodiment of the present invention provides high density lipoprotein (HDL) or reconstituted HDL (rHDL) particles for the delivery of therapeutic agents to cells and tissues. Specifically excluded from such therapeutic agents are nucleic acid based therapeutic agents, such as an oligonucleotide, a RNA, a DNA, a siRNA, a shRNA, a therapeutic gene, gene therapy vectors, and so forth. The use of HDL or rHDL particles has advantages over other delivery systems because they are smaller in size and their contents are rapidly internalized by receptors of specific cells, including receptors on the surface of tumor tissue. Methods of delivering therapeutic agents to cells and target tissues using the disclosed HDL or rHDL particles are provided as are methods of treating various diseases and disorders.

### BRIEF DESCRIPTION OF THE DRAWINGS

FIG. 1. Schematic depiction of 1,2-dimyristoyl-sn-glycero-3-phosphoethanolamine-N-[methoxy(polyethylene glycol)-2000] ammonium salt (DMPMP).

FIG. 2. Effects of DMPMP (PEG-PE) in an siRNA rHDL formulation. The inclusion of DMPMP resulted in reduction of the polydispersity index.

FIG. 3. Transmission electron microscopy of a DMPMP containing rHDL formulation.

FIG. 4. Stabilization of siRNA containing rHDL nanoparticles via lyophilization in the presence of cryoprotectants.

FIG. 5. Quantitative recovery of the siRNA from stored rHDL/siRNA formulations (6 months storage at −20° C.). of the powdered form.

FIG. 6. Double fluorescent labeling of the siRNA (Alexa488-siRNA) and of the lysozomes (Lysotracker Red) was used to illustrate that there was no overlap of the respective staining patterns. This indicates that siRNA delivery is endosome-independent.

FIG. 7. Percentage Retention of Doxorubicin in rHDL-Dox nanoparticles prepared using Triethyl and Diethyl Amine with Initial Dox concentration 0.75 mg/ml. About twice as much triethylamine (TEA) doxorubicin was retained within the rHDL formulations after 72 hours as compared to diethylamine (DEA) doxorubicin.

FIG. 8. Doxorubicin contained within rHDL are considerably less toxic to normal cells as compared to liposomal formulations of doxorubicin.

FIG. 9. Comparison of the survival of high (TC-32) and low (TC-71) SR-B1 receptor expressing EWS cells, treated with rHDL-DOX nanoparticles. TC-71 cells had a higher survival rate.

FIG. 10. Illustration of Doxorubicin uptake in the presence of anti-SR-B1 antibodies. The antibodies inhibited uptake of rHDL particles containing doxorubicin.

FIGS. 11A-11B and FIG. 12 demonstrate that doxorubicin is released into cells. Alexa 647-conjugated Apo A-I (red; FIG. 11B)) only localized in the membrane of the cell and does not enter the cell. In contrast, doxorubicin (green) is released into the cell cytoplasm (see FIG. 11A). FIG. 12 further illustrates the transfer of doxorubicin into the cells after release from the rHDL nanoparticles.

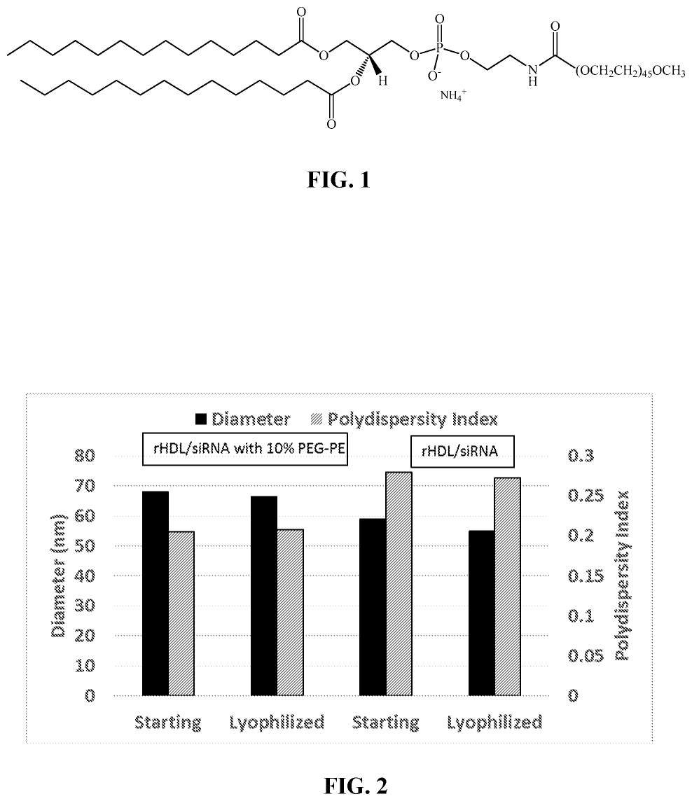

### DETAILED DISCLOSURE OF THE INVENTION

### High Density Lipoprotein (HDL) Particles or Reconstituted HDL (RHDL) Particles for the Delivery of Nucleic Acids and Other Therapeutic Agents

### Definitions

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value that varies from the numerical value set forth in this disclosure by ±10% or less. Thus, for any given numerical value, the variation may be ±1, ±2, ±3, ±4, ±5, ±6, ±7, ±8, ±9, or ±10%.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Further, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, include the phrases “consisting essentially of”, “consists essentially of”, “consisting”, and “consists”. The phrases “consisting essentially of” or “consists essentially of” indicate that the claim encompasses embodiments containing the specified materials or steps and those that do not materially affect the basic and novel characteristic(s) of the claim.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of a nucleic acid that inhibits the expression of a gene that encodes an MMP and a neutral lipid for the purposes of minimizing the growth or invasion of a tumor. The terms “treatment”, “treat”, “treating”, and grammatical variants thereof, refer to the palliation or reduction in the frequency or severity of the signs or symptoms of a disease.

A “subject” refers to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of a condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

A “disease” or “health-related condition” can be any pathological condition of a body part, an organ, or a system resulting from any cause, such as infection, genetic defect, and/or environmental stress. The cause may or may not be known.

The term “polydisperity index” (PDI), as used herein, means the distribution of particle sizes in a particulate sample; a measure of the heterogeneity of nanoparticle size and aggregation. In Dynamic Light Scattering (DLS) the size distribution of molecules or particles is the property of interest and the distribution describes the presence of material in different size “slices.” In DLS, the native distribution is the intensity distribution which indicates how much light is scattered from the various size “slices” or “bins.”

The values for different classes of dispersity are listed in the Table below.

Approximate values for dispersity parameters

| | | Distribution Type | | | |
|---|---|---|---|---|---|
| | | “monodisperse” | | “polydisperse” | |
| Parameter | Definition | uniform | narrow | moderate | broad |
| PDI from DLS | =(width/mean)² | 0.0 | 0.0-0.1 | 0.1-0.4 | >0.4 |

The moderate column indicates an intermediate, moderately polydisperse distribution type, where the distribution is neither extremely polydisperse, or broad, nor in any sense narrow.

### Lipid Binding Proteins

The term “lipid binding protein,” as used here, refers to synthetic or naturally occurring peptides or proteins that are able to sustain a stable complex with lipid surfaces and thus able to function to stabilize the lipid monolayer of the nanoparticle of the invention. The HDL or rHDL particles of the present invention may include one or more types of lipid binding proteins or apolipoproteins that are natural components of plasma lipoproteins (Ajees et al., 2006). In some embodiments, nanoparticles can be prepared using small synthetic peptides that may serve as surrogates for apo A-I (Navab et al., 2005) and thus yield formulations with additional properties once incorporated into the HDL or rHDL particles of the present invention.

### Modified Lipid Binding Proteins

Apolipoproteins generally include a high content of amphipathic motif that facilitates their ability to bind to hydrophobic surfaces, including lipids. An important characteristic of apolipoptoteins is to support the structure of monolayers, vesicles or bilayers, composed primarily of phospholipids and to transform them into disc-shaped complexes (Saito et al., 2004). Subsequently, under physiological conditions, the discoidal complexes undergo a transition to a spherical structure (Alexander et al., 2005), facilitated by the enzyme lecithin cholesterol acyltransfetase (LCAT) to produce HDL.

### Modified Lipid Binding Polypeptides

In some embodiments of the invention, a lipid binding protein (apo A-I) is used following chemical modification so that when the modified apo A-I is used as a component of the drug carrying delivery particle, it will have increased targeting ability. In one example, the apo A-I protein is modified by the attachment of folic acid residues that results in the doubling of the drug uptake by ovarian cancer cells compared to the non-modified formulation.

### Targeting Ligands

The delivery particle of the invention may include a targeting ligand bound to the lipid binding protein component. For example, Apo A-I is the natural ligand for the HDL receptors. This receptor system allows the selective uptake of the natural core component, cholesteryl ester from HDL. Studies have demonstrated that the drug paclitaxel is also taken up by cancer cells via this receptor mediated mechanism, when encapsulated by HDL delivery particles (Lacko et al., 2002).

In some embodiments involving the treatment of malignant tissues, targeting is a major advantage because most cancerous growths have been shown to have enhanced receptor expression and thus would favor the uptake of the drug that is encased in the delivery particles compared to normal tissues and thus would reduce the danger of side effects.

In other embodiments, additional receptor binding components may be attached to a lipid binding protein component to enhance the targeting potential of the delivery vehicle. In one embodiment, folate is attached to the lipid binding protein. Folate receptors are upregulated in most ovarian tumors. Because nearly all cancer cells feature substantially higher expression of one or more specific surface antigens, ultimately individual therapy of patients will be possible following a proteomic screen of the tumor (Calvo et al., 2005). In another embodiment, the lipid binding protein moiety of the delivery particle may be modified to produce specifically targeted therapeutic strategies.

### Additional Therapeutic Agents

The particles of the present invention may optionally include one or more additional therapeutic agents. For example, the therapeutic agent may be a chemotherapeutic agent. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ωI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristine, vinblastine and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

Particular aspects of the present invention concern particles that include an apolipoprotein or reconstituted high density lipoproteins, one or both of sphingomyelin and/or a PEG or polyethylene glycol containing phospholipid (PEG-phospholipid), including, for example, 1,2-dimyristoyl-sn-glycero-3-phosphoethanolamine-N-[methoxy(polyethylene glycol)-2000] ammonium salt (DMPMP) and a nucleic acid component (e.g., a therapeutic nucleic acid segment and a polypeptide comprising a positively-charged region) or other therapeutic agent, wherein the positively-charged region is associated with the nucleic acid component. These particles may be referred to as HDL or rHDL particles. The apolipoprotein can be any apolipoprotein, such as apolipoprotein A-I (Apo A-I), apoplipoprotein A-II (Apo A-II), apolipoprotein A-IV (apo-A-IV), apolipoprotein A-V (apo-V), apolipoprotein B48 (Apo B48), apoplipoprotein B100 (Apo B100), apolipoprotein C-I (Apo C-I), apolipoprotein C-II (Apo C-II), apolipoprotein C-III (Apo C-III), apolipoprotein C-IV, and apolipoprotein D (apoD). In specific embodiments, the apolipoprotein is Apo A-I.

PEG or polyethylene glycol containing phospholipids (also referred to as PEGylated phospholipids) that can incorporated into the HDL or rHDL particle compositions may include, for example, the 14 carbon (myristic acid) containing PEG-phosphoethanolamine (1,2-dimyristoyl-sn-glycero-3-phosphoethanolamine-N-[methoxy(polyethylene glycol)-2000] ammonium salt) (DMPMP) or, alternatively, may include, for example, the 18 carbon (stearic) containing PEG-phosphoethanolamine, the 16 carbon (palmitic) containing PEG-phosphoethanolamine, or the 18:1 (oleic) containing PEG-phosphoethanolamine. The phosphoethanolamine used may have, for example, a 2000 MW PEG component, or a 350 MW PEG component, 550 MW PEG component, 750 MW PEG component, 1000 MW PEG component, 3000 MW PEG component, 4000 MW PEG component or 5000 MW PEG component. Any of the phospholipids discussed below may be PEGylated according to methods known in the art using the aforementioned PEG components (e.g., a 2000 MW PEG component, 350 MW PEG component, 550 MW PEG component, 750 MW PEG component, 1000 MW PEG component, 3000 MW PEG component, 4000 MW PEG component, or 5000 MW PEG component). In certain embodiments the PEGylated phospholipid used to form HDL or rHDL is DMPMP.

In some embodiments, the particle comprises reconstituted high density lipoproteins. “Reconstituted high density lipoproteins” (rHDL) as used herein refer to spherical macromolecular complexes that contain at least three of the lipid components and one protein component of natural circulating HDL. Non-limiting examples of lipid components of natural circulating HDL include phosphatidylcholine, triglycerides, cholesterol, and cholesteryl ester. In particular embodiments, the lipid component includes cholesterol, cholesterol oleate, or a mixture of cholesterol and cholesterol oleate. In various preferred embodiments, one or more pegylated phospholipid, such as DMPMP, are used to formulate the HDL or rHDL particles in amounts that constitute between 0.5% and about 15% of the total phospholipid content, preferably between about 2.5% and about 15%, about 5% and about 12.5%, about 7.5% and 12.5% or about 10% of the total lipid content of a HDL or rHDL particle.

A “polypeptide” as used herein refers to a consecutive series of two or more amino acid residues. The polypeptide may have a length of 2 to 2000 consecutive amino acids, 2 to 1000 consecutive amino acids, 2 to 500 consecutive amino acids, 2 to 400 consecutive amino acids, 2 to 300 consecutive amino acids, 2 to 200 consecutive amino acids, 2 to 100 consecutive amino acids, 2 to 50 consecutive amino acids, 2 to 40 consecutive amino acids, 2 to 30 consecutive amino acids, 2 to 20 consecutive amino acids, or 2 to 15 consecutive amino acids.

A positively charged region of a polypeptide is a region that includes a net positive charge, that includes at least one positively charged amino acid. In particular embodiments, the polypeptide includes two or more consecutive positively charged amino acid residues. The positively charged region has a net positive charge, and functions to neutralize the negatively charged nucleic acid molecule, which thus facilitates packaging of the nucleic acid molecule into HDL or rHDL particles. For example, the positively charged amino acids may be lysine residues, histidine residues, arginine residues, positively charged non-natural amino acids, such as those described in U.S. Pat. No. 6,783,946, or a mixture of any of these residues. The amino acid segments can include any number of consecutive positively charged residues, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200 or more residues, or any range of residues derivable therein. In some embodiments, for example, the amino acid segment includes 2 to 40 consecutive lysine residues. In more particular embodiments, the amino acid segment comprises 2 to 40 consecutive lysine residues. In further embodiments, the amino acid segment comprises 2 to 20 consecutive lysine residues. In other embodiments, the amino acid segment comprises 2 to 15 consecutive lysine residues.

In addition to PEGylated phospholipids, such as DMPMP, and/or sphingomyelin, the HDL or rHDL particle of the present invention may, optionally, include one or more neutral phospholipid. Non-limiting examples of neutral phospholipids include phosphatidylcholine, phosphatidylethanolamine, 1,2-dioleoyl-sn-glycero-3-phosphatidylcholine (DOPC), egg phosphatidylcholine (“EPC”), dilauryloylphosphatidylcholine (“DLPC”), dimyristoylphosphatidylcholine (“DMPC”), dipalmitoylphosphatidylcholine (“DPPC”), di stearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), 1,2-di stearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), palmitoyloeoyl phosphatidylcholine (“POPC”), lysophosphatidylcholine, dilinoleoylphosphatidylcholine di stearoylphophatidylethanolamine (“DSPE”), dimyristoyl phosphatidylethanolamine (“DMPE”), dipalmitoyl phosphatidylethanolamine (“DPPE”), palmitoyloeoyl phosphatidylethanolamine (“POPE”), and lysophosphatidylethanolamine.

The HDL and rHDL particles disclosed herein may further comprise phospholipids. In certain embodiments, a single kind or type of phospholipid may be used in the creation of lipid compositions such as liposomes (e.g., DOPC used to generate neutral liposomes). In other embodiments, more than one kind or type of phospholipid may be used. Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DWG”), dipalmitoylphosphatidylglycerol (“DPPG”), distearoylphosphatidylglycerol (“DSPG”), distearoyl sphingomyelin (“DSSP”), di stearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine. Phospholipids include, for example, phosphatidylcholines, phosphatidylglycerols, and phosphatidylethanolamines; because phosphatidylethanolamines and phosphatidylcholines are non-charged under physiological conditions (i.e., at about pH 7), these compounds may be particularly useful for generating neutral liposomes. In certain embodiments, the phospholipid DOPC is used to produce non-charged liposomes or lipid compositions.

The HDL or rHDL particle can be of any size, but in particular embodiments the particle has a molecular size of from about 40 to about 80 nanometers, preferably about 50 to about 80 nanometers. The size may be dependent on the size of the nucleic acid component incorporated into the particle. In other embodiments, the HDL or rHDL particles have a polydispersity index of less than 0.4, and more preferably less than 0.3. In other embodiments, the polydispersity index is between about 0.01 and about 0.4, preferably between about 0.1 and about 0.3, more preferably between 0.15 and about 0.3, and even more preferably between about 0.2 and about 0.3.

The HDL or rHDL particles disclosed herein also demonstrate enhanced long-term stability. The HDL and rHDL particles of the invention can be lyophilized into a dry powder for long term storage and are stable in solution for a period of at least 60 days at 4° C.

The nucleic acid component may include any type of therapeutic nucleic acid. For example, the therapeutic nucleic acid may be nucleic acid that encodes a therapeutic agent, such as a protein. The therapeutic nucleic acid may inhibit the expression of a gene. The nucleic acid component may be a DNA or a RNA. The nucleic acid component may be an oligonucleotide of between about 2 to about 100 nucleobases in length, or it may be a polynucleotide of greater than 100 nucleobases in length. In specific embodiments, the nucleic acid component includes an interference RNA. For example, the interference RNA may be a siRNA, or a nucleic acid encoding a siRNA. For example, the siRNA may be a double-stranded nucleic acid of about 18 to about 100 nucleobases in length. In specific embodiments, the siRNA is 18 to 30 nucleobases in length. In certain embodiments, the nucleic acid component includes a shRNA or a nucleic acid encoding a shRNA.

In various embodiments, the HDL or rHDL particle further includes one or more attached ligands to target the particle to a particular cell type or tissue type in a subject. The targeting ligand can be attached to the particle using any method known to those of ordinary skill in the art. In specific embodiments, the targeting ligand is attached to the protein component of the apolipoprotein by a covalent bond. Non-limiting types of targeting ligands include a small molecule, a peptide, a polypeptide, a protein, an antibody, or an antigen binding antibody fragment. In some embodiments, the targeting ligand targets the particle to a tumor cell.

Further variation in compositional properties of the lipids can readily be achieved by introducing phosphoglycerides with a desired composition or employing other lipids (e.g., cationic lipids) when preparing the HDL-lipid or rHDL-lipid mix. Alteration of surface properties by chemical modification of lipids or apolipoproteins may also be used to alter the specificity of tissue delivery and to enhance the effectiveness of therapies designed for targeting specific metastatic tumors. Because circulating rHDL and HDL may contain apolipoproteins (A-II, A-IV, C-I, C-II, E and F), other than apo-AI, addition of these alone or in combination may be used to enhance specificity of delivery to certain types of metastatic tumors. Peptide analogs of these apolipoproteins may also be employed in the design of specific HDL or rHDL preparations as described for apo-A1.

The HDL or rHDL particles of the present invention may include a therapeutic agent, such as a chemotherapeutic agent, a single therapeutic nucleic acid, more than one therapeutic nucleic acid or a combination thereof. Thus, the particles of the present invention may comprise one or more therapeutic agent incorporated into the particle, which may or may not be a nucleic acid. Non-limiting examples of therapeutic agents include small molecules, a peptide, a polypeptide, a protein, an antibody, and/or an antigen binding antibody fragment.

Also disclosed are pharmaceutical compositions that include any of the aforementioned HDL-nucleic acid particles or rHDL-nucleic acid particles and one or more pharmaceutically acceptable carriers. The carrier can be any pharmaceutically acceptable carrier. In specific embodiments, the carrier is an aqueous carrier. Non-limiting examples of aqueous carriers include water and saline. The pharmaceutical composition may, additionally, contain an apolipoprotein, a nucleic acid component comprising a therapeutic nucleic acid segment, and a polypeptide that includes a positively-charged region, wherein the positively-charged region is associated with the nucleic acid component, are also contemplated by the present invention.

The phrases “pharmaceutical or pharmacologically acceptable” refers to molecular entities and compositions that do not produce an adverse, allergic or other untoward reaction when administered to an animal, such as a human, as appropriate. For animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is preferably formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

The pharmaceutical compositions set forth herein may further include one or more therapeutic agents. The therapeutic agent may be any therapeutic agent known to those of ordinary skill in the art, such as a small molecule, a peptide, a polypeptide, a protein, an antibody, an antigen binding antibody fragment, an oligonucleotide, a RNA, a DNA, a siRNA, a shRNA, and so forth. In particular embodiments, the pharmaceutical composition may one or more chemotherapeutic agents. Non-limiting examples of chemotherapeutic agents are set forth in the specification below.

Also disclosed are methods of treating a subject with a disease that involves administering to the subject a pharmaceutically effective amount of any of the aforementioned compositions that include a HDL-nucleic acid particle or rHDL-nucleic acid particle of the present invention. In subject can be any subject, such as a mouse, a rat, a rabbit, a cat, a dog, a cow, a horse, a sheep, a goat, a primate, or a human. In specific embodiments, the subject is a human, such as a human in need of a therapeutic nucleic acid.

The disease to be treated can be any disease known to those of ordinary skill in the art which may be amenable to treatment with a therapeutic nucleic acid. For example, the disease may be a hyperproliferative disease, an infectious disease, an inflammatory disease, a degenerative disease, or an immune disease. In particular embodiments, the hyperproliferative disease is a disease associated with neovascularization. In more particular embodiments, the hyperproliferative disease is cancer. The cancer can be any type of cancer. For example, the cancer may be breast cancer, lung cancer, prostate cancer, ovarian cancer, brain cancer, liver cancer, cervical cancer, pancreatic cancer, colon cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

The methods set forth herein may further involve the administration of one or more additional therapies to the subject. The type of therapy is largely dependent on the type of disease which is being treated. For example, where the disease is cancer, the additional therapy may be an anticancer therapy, such as a chemotherapeutic agent, radiation therapy, surgical therapy, immunotherapy, gene therapy, or a combination of these therapies. Non-limiting examples of chemotherapeutic agents include docetaxel, paclitaxel, chlorambucil, gencitabine, 6-thioguanine, mercaptupurine, methotrexate, cisplatin, oxaliplatin, carboplatin, vinbastine, etoposide, vincristine, daunomycin, capecitabine, procarbazine, mechlorethamine, cyclophosphamide, camptothecin, bleomycin, busulfan, dactinomycin, tamoxifen, raloxifene, and 5-fluorouracil.

The pharmaceutical compositions can be administered using any method known to those of ordinary skill in the art. For example, the composition may be administered to the subject intravenously, topically, locally, systemically, intraperitoneally, intratracheally, intratumorally, intramuscularly, endoscopically, intralesionally, percutaneously, subcutaneously, regionally, or by direct injection or perfusion. In specific embodiments, the composition is administered intravenously.

The present invention also concerns methods of delivering a nucleic acid segment into a cell that involves contacting the cell with an effective amount of a high density lipoprotein-nucleic acid particle of the present invention, wherein the nucleic acid segment is delivered into the cell. The cell can be any type of cell. In particular embodiments, the cell is a mammalian cell. In more particular embodiments, the cell is a tumor cell. In particular embodiments, the cell is a cell that expresses a receptor that binds to an apolipoprotein.

Also disclosed are methods of improving the therapeutic efficacy of a chemotherapeutic agent in a subject with cancer, administering to a subject with cancer a pharmaceutically effective amount of a composition of the present invention, and administering a chemotherapeutic agent to the subject, wherein efficacy of the chemotherapeutic agent is improved. Efficacy may be improved relative to a reference level of efficacy, such as efficacy with chemotherapeutic agent alone. In some embodiments, the cancer is ovarian cancer or colon cancer. In particular embodiments, the drug is a taxane, such as paclitaxel or docetaxel.

Methods of reducing the risk of metastasis in a subject with cancer that involves administration to a subject with cancer a pharmaceutically effective amount of a pharmaceutical composition of the present invention area also set forth.

Also disclosed are methods of preparing a high density lipoprotein-nucleic acid particle that involve preparing a composition which includes: (i) a polypeptide that includes a positively charged region; and (ii) a nucleic acid component that includes a therapeutic nucleic acid segment; and combining the foregoing composition with an apolipoprotein, sphingomyelin and/or PEGylated phospholipids, such as DMPMP, wherein a high density lipoprotein-nucleic acid particle is formed. The method may, optionally, include the addition of one or more neutral phospholipid (discussed above) or components that form a HDL or rHDL particle in the composition that includes the polypeptide and the nucleic acid component. The neutral phospholipid may be any type of neutral phospholipid, including any of those which have been previously mentioned. In specific embodiments, the neutral phospholipid is phosphatidylcholine. In a specific embodiment, the composition that includes the polypeptide and the nucleic acid segment further includes phosphatidylcholine, cholesterol, and cholesteryl oleate (for the formation of HDL or rHDL particles).

The present invention also concerns kits which include a first sealed container that includes an apolipoprotein and a polypeptide comprising a positively-charged region as set forth above. The apolipoprotein and polypeptide can be any of those which have been discussed in the foregoing sections. In some embodiments, the first sealed container further includes a nucleic acid component that includes a therapeutic nucleic acid segment. In some embodiments, the first sealed container includes any of the aforementioned HDL-nucleic acid particles or rHDL-nucleic acid particles of the present invention. In other embodiments, the nucleic acid component is included in a second sealed container rather than the first sealed container. The nucleic acid component may be any of the aforementioned nucleic acid components. In specific embodiments, the nucleic acid component is a siRNA.

The present invention can be applied to the treatment of any disease for which delivery of a therapeutic agent, such as a nucleic acid, to a cell or tissue of a subject is believed to be of therapeutic benefit. Examples of such diseases include hyperproliferative diseases, inflammatory diseases, infectious diseases, degenerative diseases, and autoimmune diseases. In particular embodiments, the disease is cancer.

For example, a siRNA that binds to a nucleic acid may be administered to treat a cancer. The cancer may be a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus. In certain embodiments, the cancer is ovarian cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, and/or a neurodegenerative disease).

The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### High Density Lipoprotein (HDL) Particles or Reconstituted HDL (RHDL) Particles for the Delivery of Non-Nucleic Acid Therapeutic Agents

### Definitions

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value that varies from the numerical value set forth in this disclosure by ±10% or less. Thus, for any given numerical value, the variation may be ±1, ±2, ±3, ±4, ±5, ±6, ±7, ±8, ±9, or ±10%.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Further, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, include the phrases “consisting essentially of”, “consists essentially of”, “consisting”, and “consists”. The phrases “consisting essentially of” or “consists essentially of” indicate that the claim encompasses embodiments containing the specified materials or steps and those that do not materially affect the basic and novel characteristic(s) of the claim.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of a therapeutic agent that inhibits the expression of a gene that encodes an WIMP and a neutral lipid for the purposes of minimizing the growth or invasion of a tumor. The terms “treatment”, “treat”, “treating”, and grammatical variants thereof, refer to the palliation or reduction in the frequency or severity of the signs or symptoms of a disease.

A “subject” refers to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of a condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

A “disease” or “health-related condition” can be any pathological condition of a body part, an organ, or a system resulting from any cause, such as infection, genetic defect, and/or environmental stress. The cause may or may not be known.

The term “polydisperity index” (PDI), as used herein, means the distribution of particle sizes in a particulate sample; a measure of the heterogeneity of nanoparticle size and aggregation. In Dynamic Light Scattering (DLS) the size distribution of molecules or particles is the property of interest and the distribution describes the presence of material in different size “slices.” In DLS, the native distribution is the intensity distribution which indicates how much light is scattered from the various size “slices” or “bins.”

The values for different classes of dispersity are listed in the Table below.

Approximate values for dispersity parameters

| | | Distribution Type | | | |
|---|---|---|---|---|---|
| | | “monodisperse” | | “polydisperse” | |
| Parameter | Definition | uniform | narrow | moderate | broad |
| PDI from DLS | =(width/mean)² | 0.0 | 0.0-0.1 | 0.1-0.4 | >0.4 |

The moderate column indicates an intermediate, moderately polydisperse distribution type, where the distribution is neither extremely polydisperse or broad, nor in any sense narrow.

### Lipid Binding Proteins

The term “lipid binding protein,” as used here, refers to synthetic or naturally occurring peptides or proteins that are able to sustain a stable complex with lipid surfaces and thus able to function to stabilize the lipid monolayer of the nanoparticle of the invention. The HDL or rHDL particles of the present invention may include one or more types of lipid binding proteins or apolipoproteins that are natural components of plasma lipoproteins (Ajees et al., 2006). In some embodiments, nanoparticles can be prepared using small synthetic peptides that may serve as surrogates for apo A-I (Navab et al., 2005) and thus yield formulations with additional properties once incorporated into the HDL or rHDL particles of the present invention.

### Modified Lipid Binding Proteins

Apolipoproteins generally include a high content of amphipathic motif that facilitates their ability to bind to hydrophobic surfaces, including lipids. An important characteristic of apolipoptoteins is to support the structure of monolayers, vesicles or bilayers, composed primarily of phospholipids and to transform them into disc-shaped complexes (Saito et al., 2004). Subsequently, under physiological conditions, the discoidal complexes undergo a transition to a spherical structure (Alexander et al., 2005), facilitated by the enzyme lecithin cholesterol acyltransferase (LCAT) to produce HDL.

### Modified Lipid Binding Polypeptides

In some embodiments of the invention, a lipid binding protein (apo A-I) is used following chemical modification so that when the modified apo A-I is used as a component of the drug carrying delivery particle, it will have increased targeting ability. In one example, the apo A-I protein is modified by the attachment of folic acid residues that results in the doubling of the drug uptake by ovarian cancer cells compared to the non-modified formulation.

### Targeting Ligands

The delivery particle of the invention may include a targeting ligand bound to the lipid binding protein component. For example, Apo A-I is the natural ligand for the HDL receptors. This receptor system allows the selective uptake of the natural core component, cholesteryl ester from HDL. Studies have demonstrated that the drug paclitaxel is also taken up by cancer cells via this receptor mediated mechanism, when encapsulated by HDL delivery particles (Lacko et al., 2002).

In some embodiments involving the treatment of malignant tissues, targeting is a major advantage because most cancerous growths have been shown to have enhanced receptor expression and thus would favor the uptake of the therapeutic agent that is encased in the delivery particles compared to normal tissues and thus would reduce the danger of side effects.

In other embodiments, additional receptor binding components may be attached to a lipid binding protein component to enhance the targeting potential of the delivery vehicle. In one embodiment, folate is attached to the lipid binding protein. Folate receptors are upregulated in most ovarian tumors. Because nearly all cancer cells feature substantially higher expression of one or more specific surface antigens, ultimately individual therapy of patients will be possible following a proteomic screen of the tumor (Calvo et al., 2005). In another embodiment, the lipid binding protein moiety of the delivery particle may be modified to produce specifically targeted therapeutic strategies.

### Therapeutic Agents

The particles of the present invention may optionally include one or more therapeutic agents within the particles or within a pharmaceutical composition containing the particles. For example, the therapeutic agent may be a chemotherapeutic agent. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ΩI1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, triethylamine doxorubicin, and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristine, vinblastine and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog).

Particular aspects of the present invention concern particles that include an apolipoprotein or reconstituted high density lipoproteins, one or both of sphingomyelin and/or a PEG or polyethylene glycol containing phospholipid (PEG-phospholipid), including, for example, 1,2-dimyristoyl-sn-glycero-3-phosphoethanolamine-N-[methoxy(polyethylene glycol)-2000] ammonium salt (DMPMP) and therapeutic agent. These particles may be referred to as HDL or rHDL particles. The apolipoprotein can be any apolipoprotein, such as apolipoprotein A-I (Apo A-I), apoplipoprotein A-II (Apo A-II), apolipoprotein A-IV (apo-A-IV), apolipoprotein A-V (apo-V), apolipoprotein B48 (Apo B48), apoplipoprotein B100 (Apo B100), apolipoprotein C-I (Apo C-I), apolipoprotein C-II (Apo C-II), apolipoprotein C-III (Apo C-III), apolipoprotein C-IV, and apolipoprotein D (apoD). In specific embodiments, the apolipoprotein is Apo A-I.

PEG or polyethylene glycol containing phospholipids (also referred to as PEGylated phospholipids) that can be incorporated into the HDL or rHDL particle compositions may include, for example, the 14 carbon (myristic acid) containing PEG-phosphoethanolamine (1,2-dimyristoyl-sn-glycero-3-phosphoethanolamine-N-[methoxy(polyethylene glycol)-2000] ammonium salt) (DMPMP) or, alternatively, may include, for example, the 18 carbon (stearic) containing PEG-phosphoethanolamine, the 16 carbon (palmitic) containing PEG-phosphoethanolamine, or the 18:1 (oleic) containing PEG-phosphoethanolamine. The phosphoethanolamine used may have, for example, a 2000 MW PEG component, or a 350 MW PEG component, 550 MW PEG component, 750 MW PEG component, 1000 MW PEG component, 3000 MW PEG component, 4000 MW PEG component or 5000 MW PEG component. Any of the phospholipids discussed below may be PEGylated according to methods known in the art using the aforementioned PEG components (e.g., a 2000 MW PEG component, 350 MW PEG component, 550 MW PEG component, 750 MW PEG component, 1000 MW PEG component, 3000 MW PEG component, 4000 MW PEG component, or 5000 MW PEG component). In certain embodiments the PEGylated phospholipid used to form HDL or rHDL is DMPMP.

In some embodiments, the particle comprises reconstituted high density lipoproteins. “Reconstituted high density lipoproteins” (rHDL) as used herein refer to spherical macromolecular complexes that contain at least three of the lipid components and one protein component of natural circulating HDL. Such rHDL particles can encapsulate or contain a therapeutic agent. Non-limiting examples of lipid components of natural circulating HDL include phosphatidylcholine, triglycerides, cholesterol, and cholesteryl ester. In particular embodiments, the lipid component includes cholesterol, cholesterol oleate, or a mixture of cholesterol and cholesterol oleate. In various preferred embodiments, one or more pegylated phospholipid, such as DMPMP, are used to formulate the HDL or rHDL particles in amounts that constitute between 0.5% and about 15% of the total phospholipid content, preferably between about 2.5% and about 15%, about 5% and about 12.5%, about 7.5% and 12.5% or about 10% of the total lipid content of a HDL or rHDL particle.

A “polypeptide” as used herein refers to a consecutive series of two or more amino acid residues. The polypeptide may have a length of 2 to 2000 consecutive amino acids, 2 to 1000 consecutive amino acids, 2 to 500 consecutive amino acids, 2 to 400 consecutive amino acids, 2 to 300 consecutive amino acids, 2 to 200 consecutive amino acids, 2 to 100 consecutive amino acids, 2 to 50 consecutive amino acids, 2 to 40 consecutive amino acids, 2 to 30 consecutive amino acids, 2 to 20 consecutive amino acids, or 2 to 15 consecutive amino acids.

In addition to PEGylated phospholipids, such as DMPMP, and/or sphingomyelin, the HDL or rHDL particle of the present invention may, optionally, include one or more neutral phospholipid. Non-limiting examples of neutral phospholipids include phosphatidylcholine, phosphatidylethanolamine, 1,2-dioleoyl-sn-glycero-3-phosphatidylcholine (DOPC), egg phosphatidylcholine (“EPC”), dilauryloylphosphatidylcholine (“DLPC”), dimyristoylphosphatidylcholine (“DMPC”), dipalmitoylphosphatidylcholine (“DPPC”), di stearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), 1,2-di stearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), palmitoyloeoyl phosphatidylcholine (“POPC”), lysophosphatidylcholine, dilinoleoylphosphatidylcholine di stearoylphophatidylethanolamine (“DSPE”), dimyristoyl phosphatidylethanolamine (“DMPE”), dipalmitoyl phosphatidylethanolamine (“DPPE”), palmitoyloeoyl phosphatidylethanolamine (“POPE”), and lysophosphatidylethanolamine.

The HDL and rHDL particles disclosed herein may further comprise phospholipids. In certain embodiments, a single kind or type of phospholipid may be used in the creation of lipid compositions such as liposomes (e.g., DOPC used to generate neutral liposomes). In other embodiments, more than one kind or type of phospholipid may be used. Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DWG”), dipalmitoylphosphatidylglycerol (“DPPG”), distearoylphosphatidylglycerol (“DSPG”), distearoyl sphingomyelin (“DSSP”), di stearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine. Phospholipids include, for example, phosphatidylcholines, phosphatidylglycerols, and phosphatidylethanolamines; because phosphatidylethanolamines and phosphatidylcholines are non-charged under physiological conditions (i.e., at about pH 7), these compounds may be particularly useful for generating neutral liposomes. In certain embodiments, the phospholipid DOPC is used to produce non-charged liposomes or lipid compositions.

The HDL or rHDL particle can be of any size, but in particular embodiments the particle has a molecular size of from about 40 to about 80 nanometers, preferably about 50 to about 80 nanometers. The size may be dependent on the size or amount of therapeutic agent incorporated into the particle. In other embodiments, the HDL or rHDL particles have a polydispersity index of less than 0.4, and more preferably less than 0.3. In other embodiments, the polydispersity index is between about 0.01 and about 0.4, preferably between about 0.1 and about 0.3, more preferably between 0.15 and about 0.3, and even more preferably between about 0.2 and about 0.3.

The HDL or rHDL particles disclosed herein also demonstrate enhanced long-term stability. The HDL and rHDL particles of the invention can be lyophilized into a dry powder for long term storage and are stable in solution for a period of at least 60 days at 4° C.

In various embodiments, the HDL or rHDL particle further includes one or more attached ligands to target the particle to a particular cell type or tissue type in a subject. The targeting ligand can be attached to the particle using any method known to those of ordinary skill in the art. In specific embodiments, the targeting ligand is attached to the protein component of the apolipoprotein by a covalent bond. Non-limiting types of targeting ligands include a small molecule, a peptide, a polypeptide, a protein, an antibody, or an antigen binding antibody fragment. In some embodiments, the targeting ligand targets the particle to a tumor cell.

Further variation in compositional properties of the lipids can readily be achieved by introducing phosphoglycerides with a desired composition or employing other lipids (e.g., cationic lipids) when preparing the HDL-lipid or rHDL-lipid mix. Alteration of surface properties by chemical modification of lipids or apolipoproteins may also be used to alter the specificity of tissue delivery and to enhance the effectiveness of therapies designed for targeting specific metastatic tumors. Because circulating rHDL and HDL may contain apolipoproteins (A-II, A-IV, C-I, C-II, E and F), other than apo-AI, addition of these alone or in combination may be used to enhance specificity of delivery to certain types of metastatic tumors. Peptide analogs of these apolipoproteins may also be employed in the design of specific HDL or rHDL preparations as described for apo-A1.

The HDL or rHDL particles of the non-nucleic acid embodiments may include more than one therapeutic agent; however, specifically excluded from such therapeutic agents are nucleic acids, such as an oligonucleotide, a RNA, a DNA, a siRNA, a shRNA, a therapeutic gene, gene therapy vectors and so forth. Thus, the particles of the non-nucleic acid embodiments may comprise one or more therapeutic agent incorporated into the particle selected from small molecules, a peptide, a polypeptide, a protein, an antibody, and/or an antigen binding antibody fragment.

Also disclosed are pharmaceutical compositions that include any of the aforementioned HDL-therapeutic agent containing particles or rHDL-therapeutic agent containing particles and one or more pharmaceutically acceptable carriers. The carrier can be any pharmaceutically acceptable carrier. In specific embodiments, the carrier is an aqueous carrier. Non-limiting examples of aqueous carriers include water and saline.

The phrases “pharmaceutical or pharmacologically acceptable” refers to molecular entities and compositions that do not produce an adverse, allergic or other untoward reaction when administered to an animal, such as a human, as appropriate. For animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is preferably formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

The pharmaceutical compositions set forth herein may further include one or more therapeutic agents. The therapeutic agent may be any therapeutic agent known to those of ordinary skill in the art, such as a small molecule, a peptide, a polypeptide, a protein, an antibody, an antigen binding antibody fragment, or chemotherapeutic agent and so forth. In particular embodiments, the pharmaceutical composition may one or more chemotherapeutic agents. Non-limiting examples of chemotherapeutic agents are set forth in the specification below.

Also disclosed are methods of treating a subject with a disease that involves administering to the subject a pharmaceutically effective amount of any of the aforementioned compositions that include a HDL particle or rHDL particle containing one or more therapeutic agent as disclosed herein. In subject can be any subject, such as a mouse, a rat, a rabbit, a cat, a dog, a cow, a horse, a sheep, a goat, a primate, or a human. In specific embodiments, the subject is a human, such as a human in need of treatment.

The disease to be treated can be any disease known to those of ordinary skill in the art which may be amenable to treatment with a therapeutic agent. For example, the disease may be a hyperproliferative disease, an infectious disease, an inflammatory disease, a degenerative disease, or an immune disease. In particular embodiments, the hyperproliferative disease is a disease associated with neovascularization. In more particular embodiments, the hyperproliferative disease is cancer. The cancer can be any type of cancer. For example, the cancer may be breast cancer, lung cancer, prostate cancer, ovarian cancer, brain cancer, liver cancer, cervical cancer, pancreatic cancer, colon cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

The methods set forth herein may further involve the administration of one or more additional therapies to the subject. The type of therapy is largely dependent on the type of disease which is being treated. For example, where the disease is cancer, the additional therapy may be an anticancer therapy, such as a chemotherapeutic agent, radiation therapy, surgical therapy, immunotherapy, gene therapy, or a combination of these therapies. Non-limiting examples of chemotherapeutic agents include docetaxel, paclitaxel, chlorambucil, gencitabine, 6-thioguanine, mercaptupurine, methotrexate, cisplatin, oxaliplatin, carboplatin, vinbastine, etoposide, vincristine, daunomycin, capecitabine, procarbazine, mechlorethamine, cyclophosphamide, camptothecin, bleomycin, busulfan, dactinomycin, tamoxifen, raloxifene, and 5-fluorouracil.

The pharmaceutical compositions can be administered using any method known to those of ordinary skill in the art. For example, the composition may be administered to the subject intravenously, topically, locally, systemically, intraperitoneally, intratracheally, intratumorally, intramuscularly, endoscopically, intralesionally, percutaneously, subcutaneously, regionally, or by direct injection or perfusion. In specific embodiments, the composition is administered intravenously.

The present invention also concerns methods of delivering a therapeutic agent into a cell that involves contacting the cell with an effective amount of a high density lipoprotein particle of the present invention, wherein the therapeutic agent is delivered into the cell. The cell can be any type of cell. In particular embodiments, the cell is a mammalian cell. In more particular embodiments, the cell is a tumor cell. In particular embodiments, the cell is a cell that expresses a receptor that binds to an apolipoprotein.

Also disclosed are methods of improving the therapeutic efficacy of a chemotherapeutic agent in a subject with cancer, administering to a subject with cancer a pharmaceutically effective amount of a composition of the present invention, and administering a chemotherapeutic agent to the subject, wherein efficacy of the chemotherapeutic agent is improved. Efficacy may be improved relative to a reference level of efficacy, such as efficacy with chemotherapeutic agent alone. In some embodiments, the cancer is ovarian cancer or colon cancer. In particular embodiments, the drug is a taxane, such as paclitaxel or docetaxel.

Methods of reducing the risk of metastasis in a subject with cancer that involves administration to a subject with cancer a pharmaceutically effective amount of a pharmaceutical composition of the present invention are also set forth.

Also disclosed are methods of preparing a high density lipoprotein particle that involve preparing a composition which includes: a therapeutic agent composition with an apolipoprotein, sphingomyelin and/or PEGylated phospholipids, such as DMPMP, wherein a high density lipoprotein-therapeutic agent containing particle is formed. The method may, optionally, include the addition of one or more neutral phospholipid (discussed above) or components that form a HDL or rHDL particle in the composition that includes the therapeutic agent. The neutral phospholipid may be any type of neutral phospholipid, including any of those which have been previously mentioned. In specific embodiments, the neutral phospholipid is phosphatidylcholine. In a specific embodiment, the composition that includes the therapeutic agent further includes phosphatidylcholine, cholesterol, and cholesteryl oleate (for the formation of HDL or rHDL particles).

The present invention also concerns kits which include a first sealed container that includes an apolipoprotein and a polypeptide as set forth above. The apolipoprotein and polypeptide can be any of those which have been discussed in the foregoing sections. In some embodiments, the first sealed container further includes a therapeutic agent. In some embodiments, the first sealed container includes any of the aforementioned HDL particles or rHDL particles of the present invention. In other embodiments, the therapeutic agent is included in a second sealed container rather than the first sealed container.

The present invention can be applied to the treatment of any disease for which delivery of a therapeutic agent to a cell or tissue of a subject is believed to be of therapeutic benefit. Examples of such diseases include hyperproliferative diseases, inflammatory diseases, infectious diseases, degenerative diseases, and autoimmune diseases. In particular embodiments, the disease is cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, and/or a neurodegenerative disease).

The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

All patents, patent applications, provisional applications, and publications referred to or cited herein are incorporated by reference in their entirety, including all figures and tables, to the extent they are not inconsistent with the explicit teachings of this specification. Following are examples which illustrate procedures for practicing the invention.

These examples should not be construed as limiting. All percentages are by weight and all solvent mixture proportions are by volume unless otherwise noted.

### EXAMPLES

Protocol for Formulating siRNA/rHDL Nanoparticles NM Ratio of 3 (siRNA/Lipid Wt/Wt Ratio 0.09 for PNI Lipid Mix_siRNA) is Provided Below.

PNI Lipid Mix_siRNA: Ionizable Lipid: DSPC:Chol:DMGPEG at 50:10:38.5:1.5 siRNA Formulation Buffer: 25 mM Sodium Acetate buffer pH 4.

### Procedure for Preparing Nanoparticles

Prepare a working stock of 230 ug/mL of siRNA in siRNA formulation buffer. The siRNA stock is at a concentration of 10 mg/mL or above when siRNA stock is in another buffer such as 10 mM Tris EDTA.

Prepare a 12.5 mM lipid Mix containing cationic lipid and other lipids. Aliquots of lipid stocks for mixing should be derived from individual lipid stocks in ethanol at concentrations above than 10 mg/mL. 10 to 20 mg/mL in ethanol is a good range for keeping individual lipid stocks stored at −4° C.

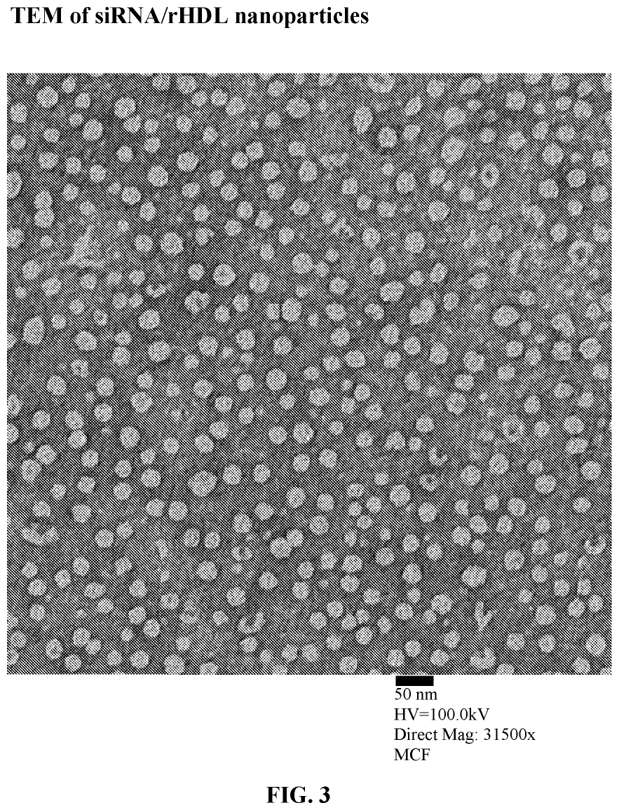

Introduce 1.5 mL of 230 ug/mL of siRNA in the siRNA formulation buffer in the left syringe and 0.5 mL of 12.5 mM of the lipid mix in ethanol in the right syringe. Insert syringes in the NanoAssemblr port. Insert two new Ambion RNase free FALCON tubes (left for collecting formulation and right for collecting waste volume). Collect the formulation. Do a size measurement immediately in PBS (15 μL of formulation to 300 uL of PBS). Dialyse the post chip formulation (12-14 K) at 4° C. overnight. Remove the formulation after dialysis and perform particle characterization studies (particle diameter, zeta potential and siRNA content (Ribogreen reagent). FIGS. 2 and 3 illustrate the characteristics of the particles.

Protocol for rHDL/siRNA Preparation Via the NanoAssemblr Instrument

All flow rates were set at 12 mL/minute and all waste volumes were collected according to manufacturer's protocol. All priming steps were followed according to manufacturer's protocol. The first step is to complex the siRNA and poly-L-lysine. A 460 μg/mL siRNA solution in Tris/EDTA buffer (10 mM Tris, 0.1 M KCl, 1 mM EDTA, pH 8.0) was put through the left channel of the instrument and mixed with a 2.3 mg/mL solution of poly-L-lysine in Tris/EDTA buffer in the right channel at a flow ratio of 1:1. The second step is to mix the siRNA/lysine complex and 10 mg/mL lipids (5 mg EYPC, 0.05 mg Cholesteryl oleate, and 0.12 mg free cholesterol in ethanol) in the left and right channels, respectively, at a flow ratio of 3:1. The last step was mixing siRNA/lysine/lipid complex and 1.67 mg Apo A-I in the left and right channels, respectively, at a flow ratio of 3:1. The formulation off-chip was dialyzed against 2 L 1×PBS at 4° C. overnight with 1 buffer change to remove residual ethanol. The formulation was then analyzed by dynamic light scattering (DLS) to determine size and polydispersity and analyzed by Ribogreen assay to determine siRNA content.

Stabilization of the siRNA Containing rHDL Nanoparticles Via Lyophilization in the Presence of Simple Carbohydrates (Cryoprotectants).

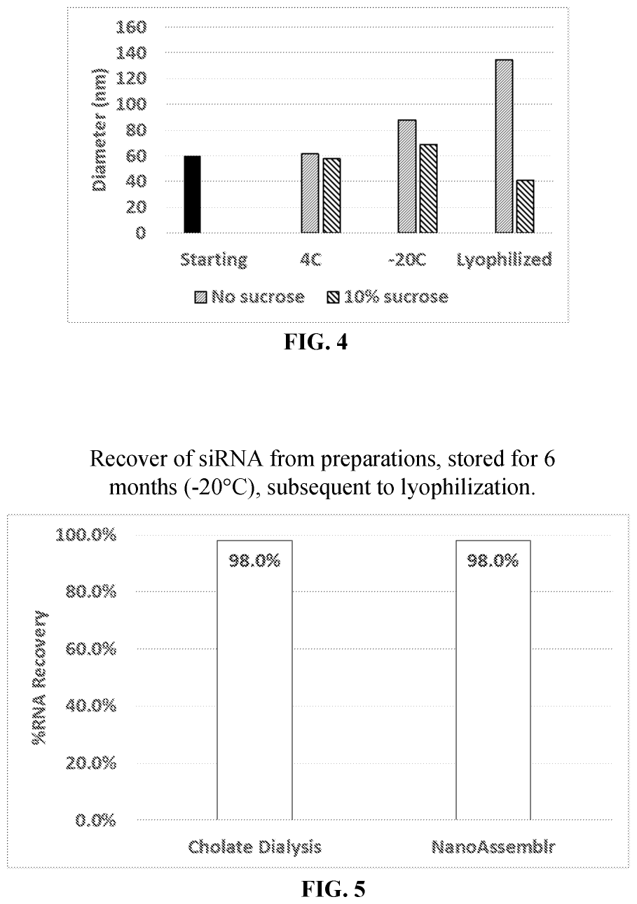

The purpose of these studies was to develop a powdered formulation of the siRNA rHDL nanoparticles that could be reconstituted without a change in their physical/chemical properties. Earlier studies indicated that the essential features of lipoprotein particles maybe preserved when sucrose (10%) was included in the preparation before lyophilization. The findings of our initial studies with this type of process are shown in FIG. 4. FIG. 5 illustrates virtually quantitative recovery of siRNA from stored rHDL/siRNA formulations (6 months storage at −20° C.) of the powdered form.

siRNA Containing rHDL Nanoparticle Uptake.

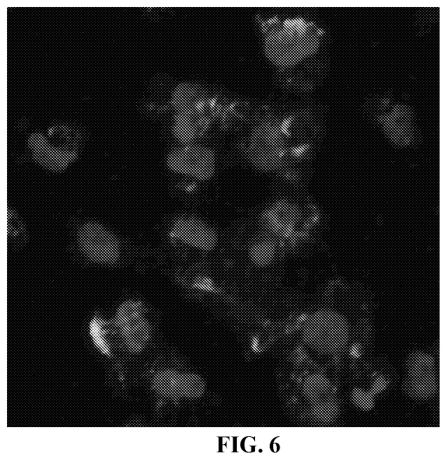

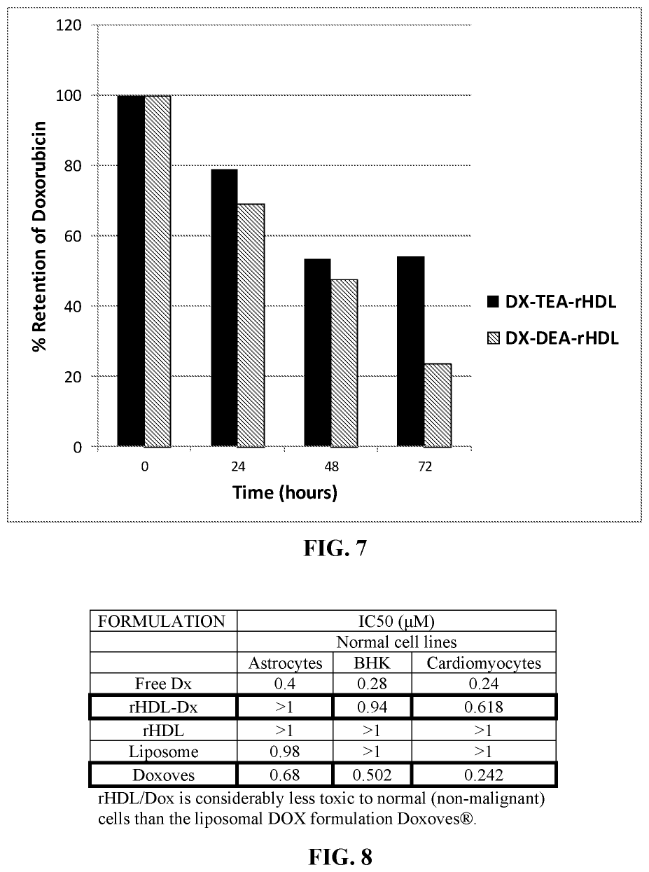

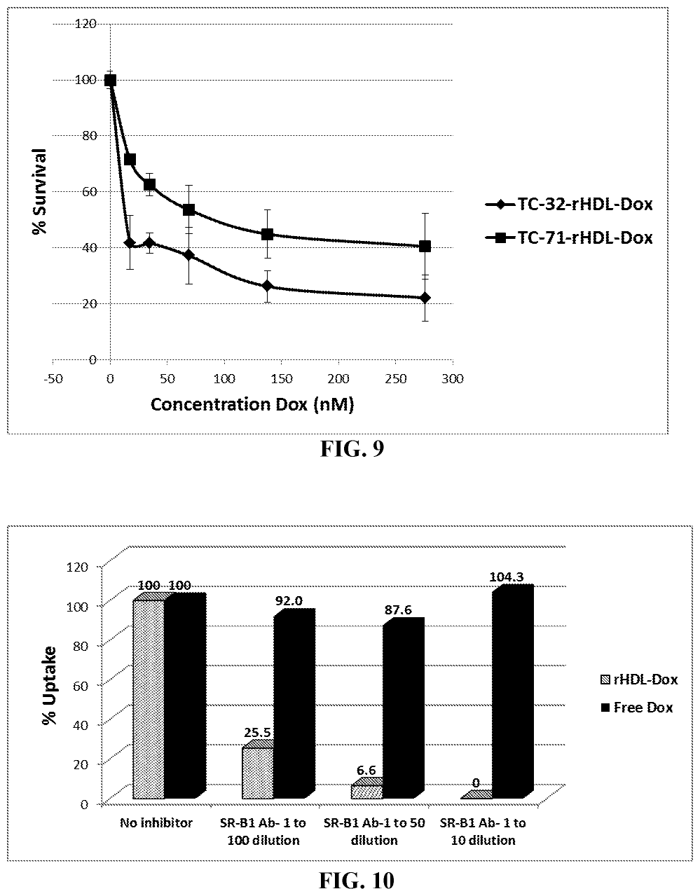

Endocytosis is a key mechanism for the uptake of needed materials for cells. The delivered material usually becomes trapped in the endosomes and is subsequently degraded by lysosomes. Thus, a major barrier to achieving effective delivery of therapeutic agents to specific cells and tissues is the endosomal/lysozomal degradative processes that could jeopardize the efficacy of the drug/agent involved. FIG. 6 illustrates that selective delivery via the SR-B1 receptor validated the endosome-independent nature of the delivery system. Double fluorescent labeling of the siRNA (Alexa488-siRNA) and of the lysozomes (Lysotracker Red), respectively, was used and no overlap of the respective staining patterns was observed. This indicates that the delivery of the siRNA to triple negative (MDA-MB-231) breast cancer cells was endosome-independent and that the nanoparticles were able to provide cytosolic delivery of siRNA to the cell.

Preparation of rHDL-Doxorubicin Formulations
1. A mixture of Egg yolk phosphatidyl choline (7.5 mg/mL), free cholesterol (0.175 mg/mL), cholesterol oleate (0.075 mg/mL) and PEG 2000 PE* (0.75 mg/mL) all in chloroform were dried down under nitrogen.
2. Doxorubicin—(0.5 mg/mL) was mixed with 10 μL Tri Ethyl Amine (TEA)** and incubated at 37° C. for 10 minutes.
3. 7.5 mg/mL sodium cholate was added and volume was made up to the desired volume with buffer (10 mM Tris, 0.1M KCl, 1 mM EDTA pH 8.0).
4. The mixture was then sonicated for 5 minutes.
5. Apo A-1* (2.5 mg/mL) was added drop wise.
6. The mixture was incubated overnight at 4° C.
7. The mixture was then dialyzed (MW cutoff 6000-8000 kD) against 1 liter of 1× phosphate buffered saline for 48 hours with change of buffer every 2 hours on the first day, (at least) 3 times and later overnight.
8. The mixture was centrifuged at 2000 rpm for 2 minutes and then sterilized by passing through 0.2 μM syringe filter.
9. The doxorubicin concentration was determined at 490 nm using a spectrophotometer.
10. Percentage Entrapment efficiency (EE) was calculated using the formula

EE={Final Doxorubicin concentration/Initial Doxorubicin}×100.

* 14:0 PEG PE1,2-dimyristoyl-sn-glycero-3-phosphoethanolamine-N-[methoxy(polyethylene glycol)-2000] (ammonium salt)**Alternatively, diethyl amine (DEA) can be used to neutralize the charge on Doxorubicin.

The delivery of therapeutic agent contained in the interior core compartment of the rHDL nanoparticles is mediated via a “selective uptake” mechanism, characteristic of the unloading of the cholesteryl ester payload from native HDL (via the SR-B1 receptor). This mechanism involves delivery of therapeutic agent to cancer cells and tumors without the endocytic uptake of the NPs and thus presents a key advantage for rHDL NPs over other nano-delivery mechanisms. By avoiding interactions with endosomes and lysozomes, the rHDL nanoparticles are protected from enzymatic and phagocytic degradation. Preliminary findings from also suggest that the rHDL NPs facilitate enhanced drug delivery and retention even by drug resistant cell lines, despite the presence of efflux pump mechanisms in these cells.

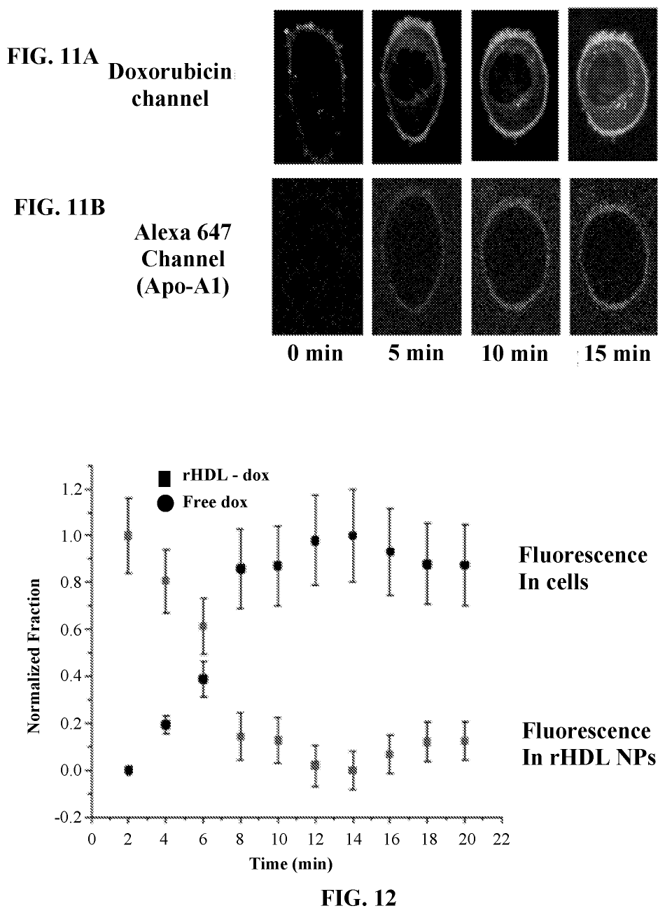

The data presented in FIGS. 11-12 were obtained via fluorescence lifetime microscopy via the following protocols. Apo A-I was labeled with Alexa Fluor 647 (Fluorescent dye Excitation: 650 nm; Emission:665 nm), using Alexa Fluor™ 647 Antibody Labeling Kit (by Thermo Fisher) according to the manufacturer's protocol. The rHDL-Doxorubicin nanoparticles were prepared with the Alexa Fluor 647 labeled Apo A-I, using the cholate dialysis protocol. The DU-145 (prostate cancer) cells were treated with the rHDL-Doxorubicin nanoparticles at 37° C./5% CO₂ and observed under confocal microscopy for uptake of doxorubicin. In order to inhibit the uptake, SR-131 antibody was used to block the receptor activity.

For imaging, the cells were plated on custom glass bottom petri dishes. Before imaging, the cells were washed with PBS, and doped with several concentrations of free or rHDL-doxorubicin formulations. For SR-B1 receptor blocking, the DU-145 cells were incubated with antibodies for SR-B1 receptor for 30 minutes at 5% CO₂ and 37° C. The cells were excited with a 470 nm diode at 70% power, and the emission imaged at 600 nm. 488 nm and a 582 nm long pass filters were placed in front of observation to better measure emission at 600 nm. Images were taken every 2 minutes. For dual label experiments, the samples were excited at 470 nm and 630 nm, with a 520 nm long pass filter on channel 1 to observe DOX fluorescence, and a 647 nm long pass on filter on channel 2 to observe ALEXA 547 emission. FIGS. 11-12 clearly demonstrate that doxorubicin is released into cells. The Alexa 647-conjugated Apo A-I (red; FIG. 11B)) only localized in the membrane of the cell and does not enter the cell. In contrast doxorubicin (green) is released into the cell cytoplasm (see FIG. 11A). FIG. 12 further illustrates the transfer of doxorubicin into the cells after release from the rHDL nanoparticles.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

U.S. Pat. No. 6,783,946
U.S. Pat. No. 8,734,853
Ajees et al., “Crystal structure of human apolipoprotein A-I: insights into its protective effect against cardiovascular diseases,” Proc. Natl. Acad. Sci. USA, 103:2126-31, 2006.
Alexander et al., “Apolipoprotein A-I helix 6 negatively charged residues attenuate lecithin-cholesterol acyltransferase (LCAT) reactivity,” Biochemistry, 44:5409-19, 2005.
Allen, “Long-circulating (sterically stabilized) liposomes for targeted drug delivery,” Trends Pharmacol. Sci., 15:215-220, 1994.
Bergers et al., “Interleukin-2-Containing Liposomes: Interaction of Interleukin-2 with Liposomal Bilayers and Preliminary Studies on Application in Cancer Vaccines,” Pharm. Res., 10:1715-1721, 1993.
Calvo et al., “Clinical proteomics: from biomarker discovery and cell signaling profiles to individualized personal therapy,” Bioscience Reports, 25:107-25, 2005.
Chonn and Cullis, “Recent advances in liposomal drug-delivery systems,” Curr. Opinion in Biotech., 6:698-708, 1995.
Fanning et al., J. Clin. Oncol., 9:1668-1674, 1993.
Feenstra et al., “Fatal hepatic coma attributed to paclitaxel,” J. Natl. Cancer Inst., 89:582-584, 1997.
Lacko et al., “High density Lipoprotein complexes as delivery vehicles for anticancer drugs,” Anticancer Res., 22:2045-9, 2002.
Lundberg, “A submicron lipid emulsion coated with amphipathic polyethylene glycol for parenteral administration of paclitaxel (Taxol),” J. Pharmacol., 49:16-21, 1997.
McGuire et al., “Cyclophosphamide and Cisplatin Compared with Paclitaxel and Cisplatin in Patients with Stage III and Stage IV Ovarian Cancer,” N. Engl. J. Med., 334:1-6, 1996.
McGuire and Ozols, “Chemotherapy of advanced ovarian cancer,” Semin. Oncol., 25:340-8, 1998.
Navab et al., “Apolipoprotein A-I mimetic peptides,” Arterioscler. Thromb. Vac. Biol., 25:1325-31, 2005.
Saito et al., “Contributions of domain structure and lipid interaction to the functionality of exchangeable human apolipoproteins,” Prog. Lipid Res., 43:350-80, 2004.
Taxi et al., “Liposomal delivery of methylphosphonate antisense oligodeoxynucleotides in chronic myelogenous leukemia,” Blood, 84:601-607, 1994.
Vingerhoeds et al., Immunoliposome-mediated targeting of doxorubicin to human ovarian carcinoma in vitro and in vivo,” Br. J. Cancer, 74:1023-9, 1996.
Wang et al., “Preparation and characterization of poly(lactic-co-glycolic acid) microspheres for targeted delivery of a novel anticancer agent taxol,” Chem. Pharm. Bull. (Tokyo), 44: 1935-40, 1996.
Weiner, “Liposomes for protein delivery: selecting manufacture and development processes,” Immunomethods 4:201-209, 1994.

1-45. (canceled)

46. A high density lipoprotein (HDL) particle or reconstituted HDL (rHDL) particle comprising: a) sphingomyelin and/or one or more PEGylated phospholipid; b) an apolipoprotein; c) a therapeutic agent or a nucleic acid component comprising a therapeutic nucleic acid segment; d) a polypeptide of 2 to 500 consecutive amino acids, the polypeptide comprising a positively-charged region associated with the nucleic acid component; and, optionally one or more neutral phospholipid,
wherein the neutral phospholipid is 1,2-dioleoyl-sn-glycero-3-phosphatidylcholine (DOPC), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), palmitoyloleoyl phosphatidylcholine (“POPC”), lysophosphatidylcholine, dilinoleoylphosphatidylcholine, distearoylphosphatidylethanolamine (“DSPE”), dimyristoyl phosphatidylethanolamine (“DMPE”), dipalmitoyl phosphatidylethanolamine (“DPPE”), palmitoyloleoyl phosphatidylethanolamine (“POPE”), lysophosphatidylethanolamine, or a combination thereof.

47. The HDL particle or rHDL particle according to claim 46 comprising:
a) sphingomyelin; an apolipoprotein; a nucleic acid component comprising a therapeutic nucleic acid segment; a polypeptide of 2 to 500 consecutive amino acids, the polypeptide comprising a positively-charged region associated with the nucleic acid component; and optionally one or more neutral phospholipid;
b) one or more PEGylated phospholipid; an apolipoprotein; a nucleic acid component comprising a therapeutic nucleic acid segment; a polypeptide of 2 to 500 consecutive amino acids, the polypeptide comprising a positively-charged region associated with the nucleic acid component; and optionally one or more neutral phospholipid; or
c) spingomyelin and one or more PEGylated phospholipid; an apolipoprotein; a nucleic acid component comprising a therapeutic nucleic acid segment; a polypeptide of 2 to 500 consecutive amino acids, the polypeptide comprising a positively-charged region associated with the nucleic acid component; and optionally one or more neutral phospholipid.

48. The HDL particle or rHDL particle of claim 46, said particle lacking one or more neutral phospholipid.

49. The HDL particle or rHDL particle of claim 46, said particle comprising one or more neutral phospholipid.

50. The HDL particle or rHDL particle of claim 46, wherein the HDL-nucleic acid particle further comprises phosphatidylcholine, triglycerides, cholesterol, cholesteryl ester, or combinations thereof and forms a reconstituted HDL (rHDL) particle.

51. The HDL particle or rHDL particle of claim 46, wherein the apolipoprotein is apolipoprotein AI.

52. A method of treating a subject with a disease comprising administering to the subject a pharmaceutically effective amount of HDL particles or rHDL particle as set forth in claim 46 or a pharmaceutical composition thereof.

53. A method of delivering a nucleic acid segment into a cell, comprising contacting the cell with an effective amount of an HDL particle or rHDL particle according to claim 46 or a pharmaceutical composition thereof.

54. A method of improving the therapeutic efficacy of a chemotherapeutic agent in a subject with cancer, comprising: a) administering to a subject with cancer a pharmaceutically effective amount of HDL particles or rHDL particle according to claim 46 or pharmaceutical compositions thereof; and b) administering a chemotherapeutic agent to the subject, wherein efficacy of the chemotherapeutic agent is improved.

55. A method of reducing the risk of metastasis in a subject with cancer, comprising: a) administering to a subject with cancer a pharmaceutically effective amount of HDL particles or rHDL particle or rHDL particle according to claim 46 or pharmaceutical compositions thereof; and b) administering a chemotherapeutic agent to the subject, wherein the risk of metastasis in the subject is reduced.

56. A high density lipoprotein (HDL) particle or reconstituted HDL (rHDL) particle comprising: a) sphingomyelin and/or one or more PEGylated phospholipid; b) an apolipoprotein; c) a therapeutic agent optionally associated with a polypeptide of 2 to 500 consecutive amino acids, and, optionally one or more neutral phospholipid,
wherein the neutral phospholipid is 1,2-dioleoyl-sn-glycero-3-phosphatidylcholine (DOPC), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), palmitoyloleoyl phosphatidylcholine (“POPC”), lysophosphatidylcholine, dilinoleoylphosphatidylcholine, distearoylphosphatidylethanolamine (“DSPE”), dimyristoyl phosphatidylethanolamine (“DMPE”), dipalmitoyl phosphatidylethanolamine (“DPPE”), palmitoyloleoyl phosphatidylethanolamine (“POPE”), lysophosphatidylethanolamine, or a combination thereof;
with the proviso that the therapeutic agent is not a nucleic acid based therapeutic agent, such as an oligonucleotide, a RNA, a DNA, a siRNA, a shRNA, therapeutic gene, a gene therapy vector, and so forth.

57. The HDL particle or rHDL particle of claim 56 comprising:
a) sphingomyelin; an apolipoprotein; a therapeutic agent; and, optionally, a polypeptide of 2 to 500 consecutive amino acids; and optionally one or more neutral phospholipid;
b) one or more PEGylated phospholipid; an apolipoprotein; a therapeutic agent; and, optionally, a polypeptide of 2 to 500 consecutive amino acids; and optionally one or more neutral phospholipid; or
c) sphingomyelin and one or more PEGylated phospholipid; an apolipoprotein; a therapeutic agent; and, optionally, a polypeptide of 2 to 500 consecutive amino acids; and optionally one or more neutral phospholipid.

58. The HDL particle or rHDL particle of claim 56, said particle lacking one or more neutral phospholipid.

59. The HDL particle or rHDL particle of claim 56, said particle comprising one or more neutral phospholipid.

60. The HDL particle or rHDL particle of claim 56, wherein the particle further comprises phosphatidylcholine, triglycerides, cholesterol, cholesteryl ester, or combinations thereof and forms a reconstituted HDL (rHDL) particle.

61. The HDL particle or rHDL particle of claim 56, wherein said therapeutic agent is triethylamine-doxorubicin.

62. A method of treating a subject with a disease comprising administering to the subject a pharmaceutically effective amount of HDL particles or rHDL particle according to claim 56 or a pharmaceutical composition thereof.

63. A method of delivering a therapeutic agent into a cell, comprising contacting the cell with an effective amount of an HDL particle or rHDL particle according to claim 56 or pharmaceutical compositions comprising said HDL or rHDL particle particles.

64. A method of improving the therapeutic efficacy of a chemotherapeutic agent in a subject with cancer, comprising: a) administering to a subject with cancer a pharmaceutically effective amount of HDL particles or rHDL particle according to claim 56 or pharmaceutical compositions thereof; and b) administering a chemotherapeutic agent to the subject, wherein efficacy of the chemotherapeutic agent is improved.

65. A method of reducing the risk of metastasis in a subject with cancer, comprising: a) administering to a subject with cancer a pharmaceutically effective amount of HDL particles or rHDL particle or rHDL particle according to claim 56 or pharmaceutical compositions thereof; and b) administering a chemotherapeutic agent to the subject, wherein the risk of metastasis in the subject is reduced.
